(19) United States
(12) Patent Application Publication
(10) Pub. No.: US 2012/0283739 A1
(43) Pub. Date: Nov. 8, 2012

(54) BONE TACK DRIVER

(76) Inventors: James D. Ralph, Bethlehem, PA (US); Thomas N. Troxell, Pottstown, PA (US); Mark Michels, Glen Mills, PA (US)

(21) Appl. No.: 13/462,928

(22) Filed: May 3, 2012

Related U.S. Application Data
(60) Provisional application No. 61/482,038, filed on May 3, 2011.
(60) Provisional application No. 61/484,526, filed on May 10, 2011.

(51) Int. Cl. A61B 17/56

(52) U.S. Cl. 606/99

(57) ABSTRACT

A driver assembly for affixing a surgical fastener to a target location is provided. Operation of the driver assembly inserts the surgical fastener in two stages, first an alignment stage through application of a distally directed force to partially insert the surgical fastener, and then a fastening stage to fully insert and seat the surgical fastener to a proper depth or compression level. The driver assembly comprises a spring loaded automatic trigger mechanism that may be adapted for use with a linearly insertable or a rotationally insertable surgical fastener. Application of the distally directed force actuates the trigger mechanism, wherein a corresponding impact force is delivered for seating the surgical fastener, coupled to a distal end of the driver assembly, upon alignment of cam and receiver elements embodied within the trigger mechanism.

### CROSS-REFERENCE TO RELATED APPLICATIONS

This application claims the benefit of U.S. Provisional Patent Application No. 61/482,038, filed May 3, 2011, and U.S. Provisional Patent Application No. 61/484,526, filed May 10, 2011, which are incorporated by reference herein in their entirety.

### TECHNICAL FIELD

Embodiments of the invention relate generally to medical devices and, more particularly, to a driver assembly for affixing a surgical fastener to a bone.

### BACKGROUND

Surgical fasteners used today include linearly insertable (i.e., push-in type) fasteners and rotationally insertable (i.e., screw-in type) fasteners. Linearly insertable surgical fasteners offer an alternative to rotationally insertable surgical fasteners, particularly in the areas of craniofacial surgery, small bone surgery and as a means for attaching or reattaching soft tissue to bone. Tacks, rivets, staples, suture anchors, plugs and soft tissue anchors are among the most common forms of linearly insertable surgical fasteners.

While linearly insertable surgical fasteners can sometimes be pushed in with a simple rigid insertion instrument, it is often desirable to insert the fastener with an impact force instead. When a linearly insertable fastener is used to provide compression (e.g. of a bone plate to a bone), an impact force will generally create more compression than simply pushing the fastener into place.

The use of small surgical fasteners is often required, particularly in craniofacial surgery, small bone surgery and arthroscopic surgery. Given their small size, the surgical fasteners can be difficult to pick-up or load onto an insertion instrument. However, it is important that surgical fasteners be properly loaded and securely fixed to the insertion instrument to avoid intraoperative complications—e.g., dislodging, misalignment or breakage of a surgical fastener during insertion.

Therefore, there exists a need for a device better adapted to handle and facilitate the insertion of surgical fasteners. More specifically, the device would allow for ease of loading and securely retaining a surgical fastener, would allow for a single-hand operation, and would reliably generate the correct impact force for proper insertion of the surgical fastener.

### BRIEF DESCRIPTION OF THE DRAWINGS

The present invention is illustrated by way of example, and not by way of limitation, and will become apparent upon consideration of the following detailed description, taken in conjunction with the accompanying drawings, in which like reference characters refer to like parts throughout, and in which:

FIGS. 1A-1C illustrate, respectively, an expanded perspective view of component parts, a cross-sectional view along a longitudinal axis and an assembled perspective view from a proximal end of an embodiment of a driver assembly adapted for use with linearly insertable surgical fasteners.

FIGS. 2A-2C illustrate, respectively, a driver shaft and tip of the driver assembly, as illustrated in FIGS. 1A-1C, having a surgical fastener loaded thereon, a conical-shaped driver tip, and a square-shaped driver tip.

FIGS. 3A and 3B illustrate, respectively, cross-sectional views along a longitudinal axis of the driver assembly, as illustrated in FIGS. 1A-1C, prior to a fully loaded release position and immediately after release of a drive spring.

FIGS. 4A and 4B illustrate, respectively, an expanded perspective view of component parts and a cross-sectional view along a longitudinal axis of an embodiment of a driver assembly adapted for use with rotationally insertable surgical fasteners.

FIGS. 5A and 5B illustrate, respectively, cross-sectional views along a longitudinal axis of the driver assembly, as illustrated in FIGS. 4A and 4B, having a snap on type driver tip and a screw on type driver tip.

FIGS. 6A and 6B illustrate, respectively, an expanded perspective view of component parts and a cross-sectional view along a longitudinal axis of an embodiment of a driver assembly adapted for use with two-part surgical fasteners.

### DETAILED DESCRIPTION

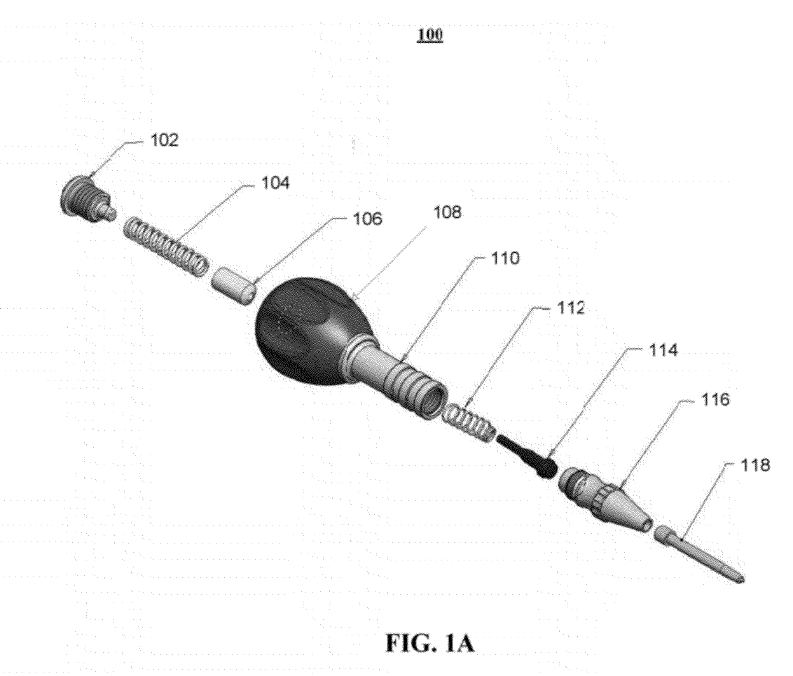

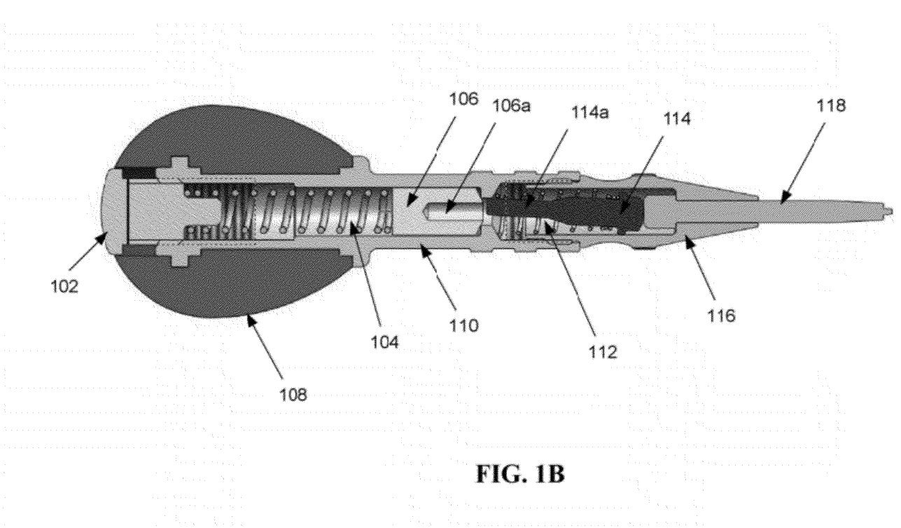

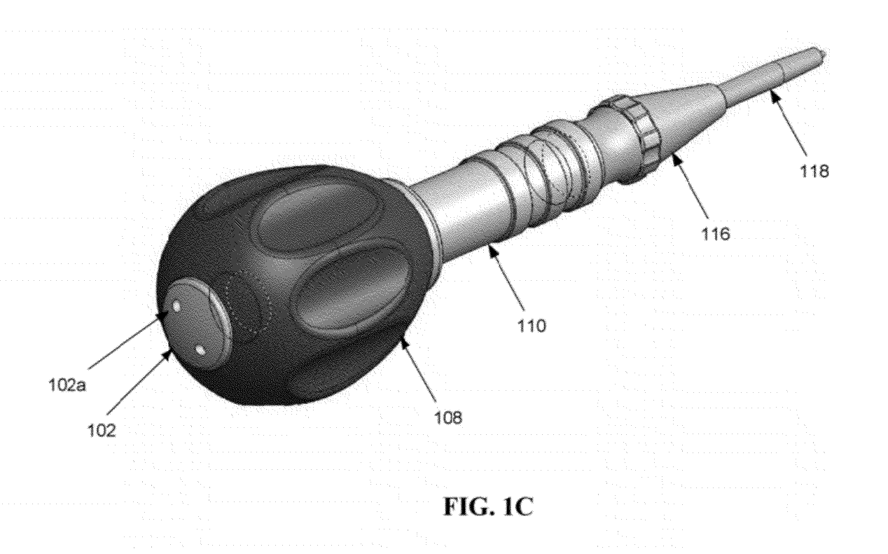

FIGS. 1A-1C illustrate, respectively, an expanded perspective view of component parts, a cross-sectional view along a longitudinal axis and an assembled perspective view from a proximal end of a driver assembly 100 adapted for use with linearly insertable surgical fasteners. Referring to FIG. 1A, driver assembly 100 may be comprised of a force adjustment screw 102, a drive spring 104, a receiver element 106, a handle portion 108, an elongated neck portion 110, an alignment spring 112, a cam element 114, a nose piece 116 and a driver shaft 118.

As illustrated in corresponding FIGS. 1B and 1C, elongated neck portion 110 may be coupled to handle portion 108, nose piece 116 may be coupled to elongated neck portion 110, and driver shaft 118 may be coupled to nose piece 116. Handle portion 108 may be constructed of a silicone rubber, or any other suitable material, molded into a body shaped to comfortably fit the hand of an operator of driver assembly 100. Driver assembly 100 itself and various components thereof may be constructed from various FDA approved material suitable for use in surgical applications.

Referring to FIG. 1B, drive spring 104 is affixed between force adjustment screw 102 and receiver element 106 embodied within elongated neck portion 110. Receiver element 106 is comprised of a bore portion 106a configured to receive a proximal end 114a of cam element 114 when centered with receiver element 106. Alignment of proximal end 114a of cam element 114 may be regulated by alignment spring 112 embodied within nose piece 116. Drive spring 104, receiver element 106, alignment spring 112 and cam element 114 may be collectively referred to herein as components of an automatic trigger mechanism. In an alternate embodiment, it is envisioned that one skilled in the art may modify elongated neck portion 110 to accommodate components of the automatic trigger mechanism in the same arrangement, as illustrated in FIG. 1B, without the need for nose piece 116. For example, elongated neck portion 110 and nose piece 116 may be unified into a single body having one or more chambers for housing components of the automatic trigger mechanism.

The amount of force required to be delivered by driver assembly 100 to firmly seat a surgical fastener may be adjusted using force adjustment screw 102 provided in handle portion 108. Force adjustment screw 102 may be comprised of apertures 102a, as illustrated in FIG. 1C, for receiving a tool to advance force adjustment screw 102 to a desired force setting. For example, a spanner wrench may be used in apertures 102a to advance force adjustment screw 102. Although illustrated as a pair of circular apertures in FIG. 1C, apertures 102a may also be modified in shape so as to be adapted to receive a hex socket wrench, a flat-head screwdriver, a Phillips-head screwdriver or any other suitable tool for advancing force adjustment screw 102 to a desired force setting. Although illustrated as a screw embodied in handle portion 108, a mechanism for adjusting a force setting of driver assembly 100 can be achieved through the use of other suitable components. Force adjustment screw 102 may be operator adjustable within a predetermined range or, alternatively, may be preset at assembly and not subject to adjustment by an operator.

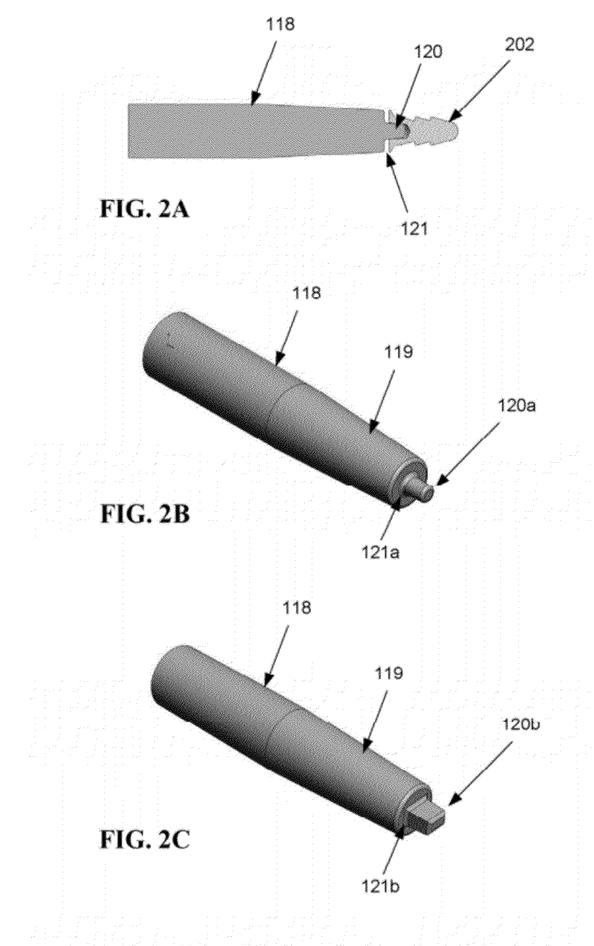

A driver tip 120 is provided, as illustrated in FIG. 2A, at a distal end of driver shaft 118 of driver assembly 100. Driver tip 120 may be any one of a plurality of tip configurations, each of which are designed to securely retain and drive a linearly insertable surgical fastener 202 into a target location of a bone. Surgical fastener 202 may be retained securely on driver tip 120 by means of a taper fit, an interference fit or any other suitable secure fastening means.

A detachable tip extension head 119 having a particular tip configuration may be coupled to driver shaft 118 to allow for ease of interchangeability between desired driver tips. For example, as illustrated in FIGS. 2B and 2C, driver tip 120 may be a conical-shaped driver tip 120a or a square-shaped driver tip 120b. Driver tip 120a and driver tip 120b may be shaped, respectively, having a shoulder area 121a and a shoulder area 121b to allow for a space 121, as illustrated in FIG. 2A, between the distal end of driver shaft 118 and a proximal end of surgical fastener 202 attached to the driver tip. To load surgical fastener 202 onto the desired driver tip 120, driver tip 120 may simply be pressed into a hole provided in the head of surgical fastener 202. Space 121 may serve to insure that a tapered driver tip inserts fully into a surgical fastener and that only the tapered driver tip is used to drive the surgical fastener. Space 121 may also serve to permit surgical fastener 202 to be easily released from driver tip 120 with a slight angular deflection of driver shaft 118.

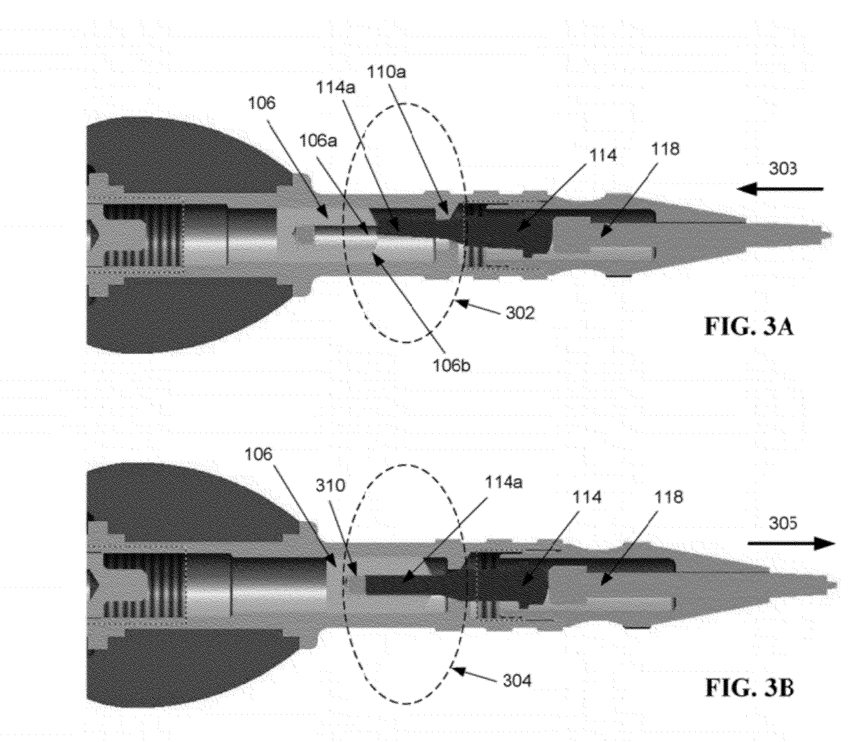

Surgical fastener 202 loaded onto driver tip 120 may be positioned, for example, through a hole in a bone plate aligned with a predrilled hole in an underlying bone. As distally directed force is applied in the direction of the target location of the bone, via handle portion 108 of driver assembly 100, drive spring 104 and alignment spring 112 undergo compression. A compression force 303, as illustrated in FIG. 3A, is returned in the proximal direction when the distally directed force is applied against the target location of the bone, wherein compression force 303 displaces driver shaft 118. Displacement of driver shaft 118 in the direction of compression force 303 pushes against and displaces cam element 114, compressing alignment spring 112 coupled thereto, which in turn pushes against and displaces receiving element 106, compressing drive spring 104 coupled thereto.

Alignment spring 112 may be configured to keep cam element 114 tilted and out of alignment with bore portion 106a, as shown in FIG. 3A, until cam element 114 is displaced to a position allowing it to be centered with bore portion 106a, as illustrated in FIG. 3B. Alignment spring 112 may also be configured to reset cam element 114 and driver shaft 118 to their original starting positions, as illustrated in FIG. 1B, prior to application of a distally directed force. As distally directed force is applied, cam element 114 is displaced in the proximal direction and an internally tapered throat 110a in elongated neck portion 110, as illustrated in section 302 of FIG. 3A, forces proximal end 114a of cam element 114 into alignment with bore portion 106a of receiver element 106. As illustrated in section 302 of FIG. 3A, the distal surface of receiver element 106 may be configured with a reverse taper end 106b to keep proximal end 114a of cam element 114 from slipping into bore portion 106a of receiver element 106 until the last possible moment.

Surgical fastener 202 loaded onto a driver tip 120 may be linearly driven into the target location of the bone as distally directed force is applied and driver shaft 118 is forced in the proximal direction. When proximal end 114a of cam element 114 is aligned with bore portion 106a of receiving element 106, as illustrated in section 304 of FIG. 3B, cam element 114 is received into bore portion 106a and the displaced receiver element 106 is driven in the distal direction by compressed drive spring 104. The resulting impact force, when the bottom of bore portion 106a makes contact with proximal end 114a of cam element 114, allows surgical fastener 202 loaded onto driver tip 120 to be driven forcefully in the distal direction, as illustrated by a driving force 305 in FIG. 3B, and further seated into the target location of the bone.

To reduce the degree of force associated with recoil resulting from delivery of driving force 305, a plug 310 may be provided in bore portion 106a of receiver element 106. Plug 310 may serve as a “dead blow” feature to soften the recoil, while still producing the desired impact, when proximal end 114a of cam element 114 is received in bore portion 106a of receiver element 106. Alternatively, receiver element 106 may be modified to include a cavity loosely filled with small pellets or spheres, similar in nature to a dead blow hammer. After surgical fastener 202 is inserted into the target location of the bone, application of a slight angular deflection of driver shaft 118 may release surgical fastener 202 from driver tip 120. As driver assembly 100 is withdrawn, drive spring 104 and alignment spring 112 are relaxed, permitting driver assembly 100 to reset itself.

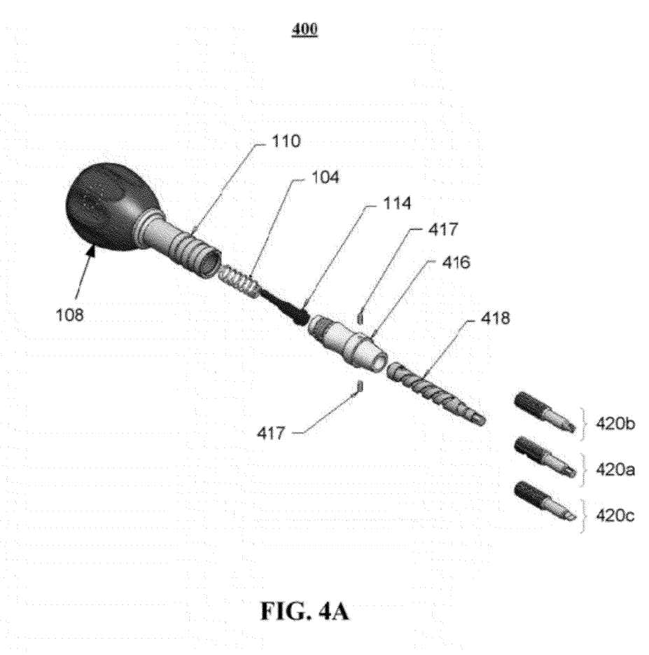

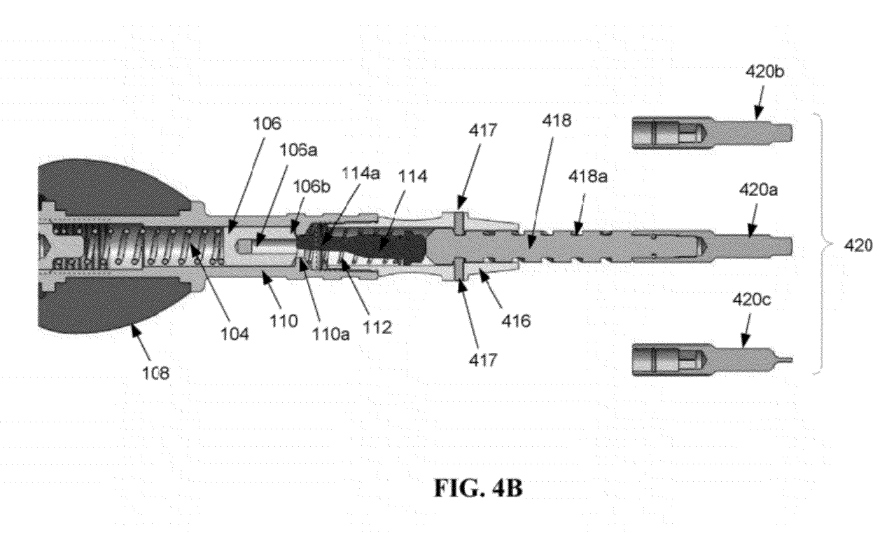

FIGS. 4A and 4B illustrate, respectively, an expanded perspective view of component parts and a cross-sectional view along a longitudinal axis of a driver assembly 400 adapted for use with rotationally insertable surgical fasteners. Referring to FIGS. 4A and 4B, driver assembly 400 is similar in construction to driver assembly 100 and may utilize the same driving mechanism, as illustrated in FIG. 1A. As in driver assembly 100, driver assembly 400 utilizes an automatic trigger mechanism comprising drive spring 104, receiver element 106, alignment spring 112 and cam element 114.

In driver assembly 400, cam element 114 and alignment spring 112 may be embodied in a nose piece 416, which is slightly modified in design from nose piece 116 in driver assembly 100 to accommodate a rotational driver shaft 418. Driver shaft 418 may be comprised of one or more helical grooves 418a provided along an exterior surface of its body to allow for a rotational movement of the shaft when force is applied to its ends. One or more pin members 417 may be positioned perpendicular to the longitudinal axis direction of driver assembly 400 through one or more apertures provided in the body of nose piece 416. The perpendicular positioning of pin members 417 provided in nose piece 416 protrude into helical grooves 418a of driver shaft 418 to enable rotational movement of driver shaft 418 about the longitudinal axis of driver assembly 400.

Similar to the application of driver assembly 100, as distally directed force is applied in the direction of a target location of a bone, via handle portion 108 of driver assembly 400, drive spring 104 and alignment spring 112 undergo compression. The distally directed force results in a rotational displacement of driver shaft 418 in a direction opposite the distally directed force, the rotational displacement pushing against and displacing cam element 114 in the proximal direction, thereby pushing against and displacing receiver element 106 communicatively coupled thereto.

The automatic trigger mechanism of driver assembly 400 operates in the same manner as previously described in connection with driver assembly 100. As distally directed force is applied, cam element 114 is displaced in the proximal direction and internally tapered throat 110a in elongated neck portion 110, as illustrated in FIG. 4B, forces proximal end 114a of cam element 114 into alignment with bore portion 106a of receiver element 106. As in driver assembly 100, the distal surface of receiver element 106 in driver assembly 400 may be configured with a reverse taper end 106b to keep proximal end 114a of cam element 114 from prematurely slipping into bore portion 106a of receiver element.

A surgical fastener loaded onto a driver tip 420 may be rotationally driven into the target location of the bone as distally directed force is applied and driver shaft 418 is forced in the proximal direction. When proximal end 114a of cam element 114 is aligned with bore portion 106a of receiving element 106, cam element 114 is received into bore portion 106a and the displaced receiver element 106 is driven in the distal direction by compressed drive spring 104. The resulting impact force further seats the surgical fastener rotationally inserted into the target location of the bone. In one embodiment, grooves 418a may terminate distally to allow for delivery of the impact force without producing any reverse rotation of driver shaft 418.

Rotational screw-type driver tips 420 may be provided, as illustrated in FIGS. 4A and 4B, at a distal end of driver shaft 418 of driver assembly 400. A plurality of tip configurations may be employed, each of which are designed to securely drive a rotationally insertable surgical fastener into a target location of a bone. Driver tips 420 may be detachable to allow for interchangeability of the desired driver tip and may be, but are not limited to, a hex driver tip 420a, a Phillip's driver tip 420b and a flat (or slot) driver tip 420c. Other types of driver tips (not shown) that may be used with driver assembly 400 may be a Frearson-type driver tip, a clutch-type driver tip, a square-type driver tip, a Bristol-type driver tip, a Torx-type driver tip, a spanner-type driver tip, a spline-type driver tip, a double hex-type driver tip, or a triple square-type driver tip.

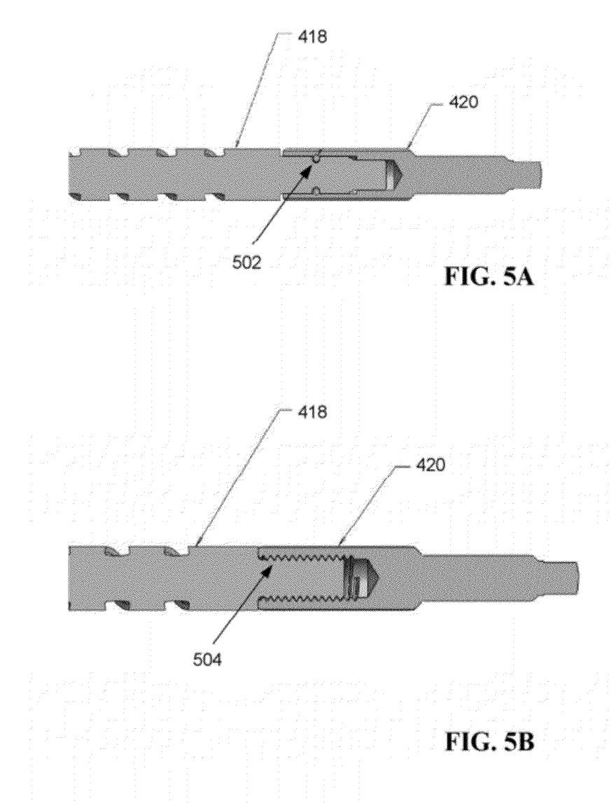

Driver tip 420 may be a snap on type driver tip, as illustrated in FIG. 5A, to allow for a secure connection with the distal end of driver shaft 404. For example, driver tip 420 may be adapted with a split locking ring 502. Alternatively, driver tip 420 may be a screw on type driver tip, as illustrated in FIG. 5B, to allow for a secure connection with the distal end of driver shaft 418. For example, driver shaft 418 and driver tip 420 may be adapted with corresponding threading 504.

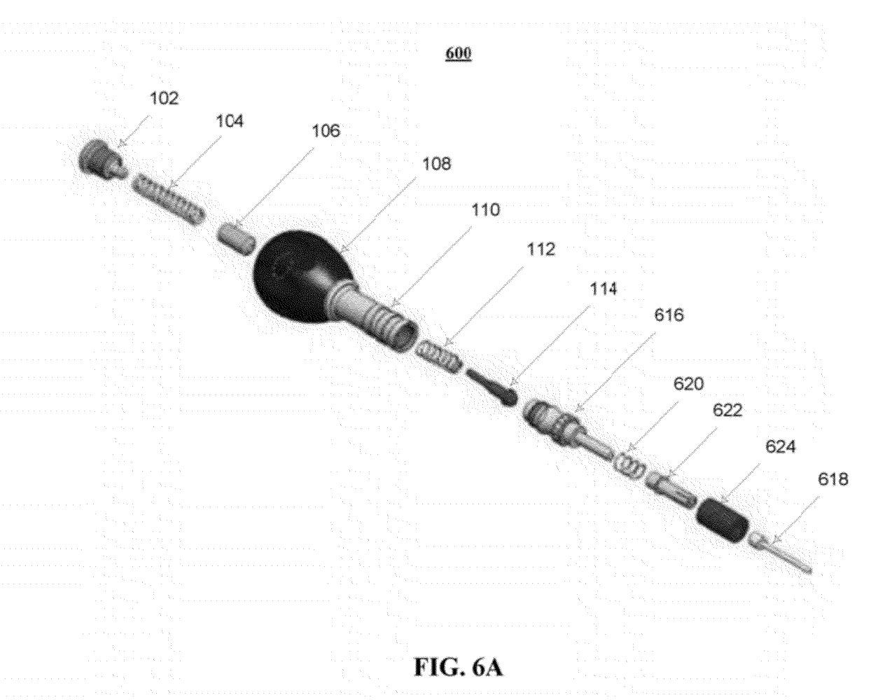

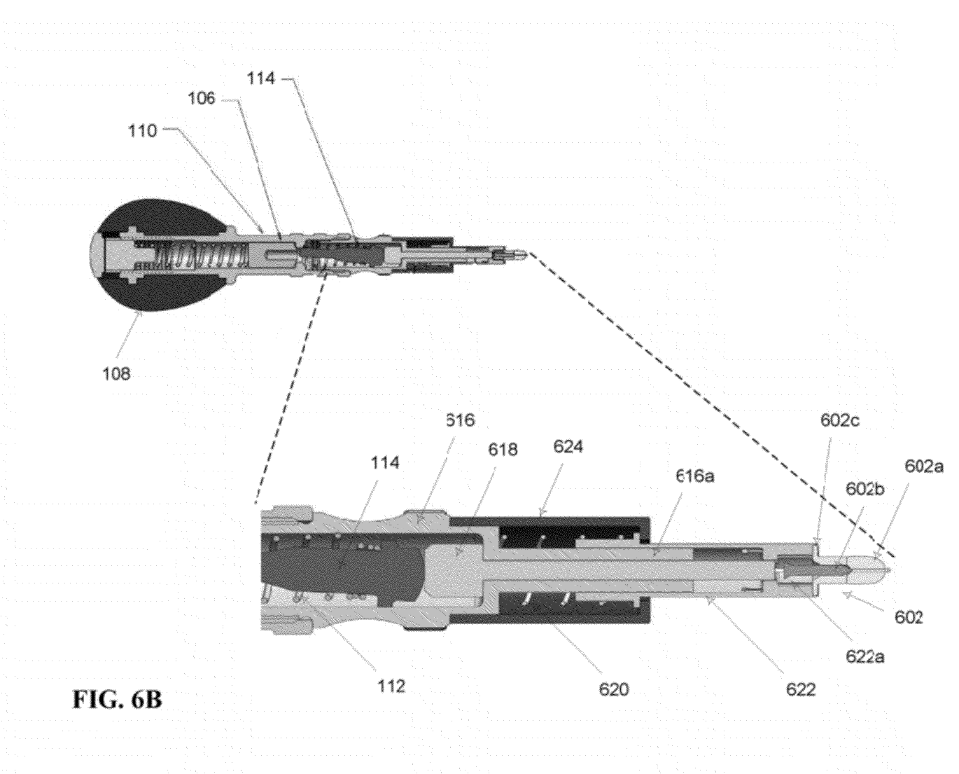

FIGS. 6A and 6B illustrate, respectively, an expanded perspective view of component parts and a cross-sectional view along a longitudinal axis of a driver assembly 600 adapted for use with a two-part surgical fastener 602. Surgical fastener 602, for example, may be comprised of an expandable outer body 602a having an internal bore to receive a central pin member 602b. As is known with expandable fasteners, when a pin member embodied within an outer body of the fastener is driven in the distal direction, the walls of the outer body may expand to create a secure interference fit.

Referring to FIGS. 6A and 6B, driver assembly 600 is similar in construction to driver assembly 100 and may utilize the same automatic trigger mechanism, as illustrated in FIG. 1A. Driver assembly 600 utilizes an automatic trigger mechanism comprising drive spring 104, receiver element 106, alignment spring 112 and cam element 114. In driver assembly 600, cam element 114 and alignment spring 112 may be embodied in a nose piece 616. Nose piece 616 may be modified in design, as compared to nose piece 116 of driver assembly 100, to further accommodate additional components comprising a front spring 620, a holding sleeve 622 and a cap member 624. In one embodiment, nose piece 616 may be configured with an elongated cylindrical portion 616a at its distal end to slidably receive front spring 620 and holding sleeve 622, which may be securely affixed to nose piece 616 by cap member 624.

Holding sleeve 622 may allow a flange portion 602c provided circumferentially along outer body 602a of surgical fastener 602 to be gripped by means of a friction, taper or interference fit, while central pin member 602b is retained within a bore provided in outer body 602a of surgical fastener 602 awaiting to be driven distally by an impact force generated by the trigger mechanism of driver assembly 600. The trigger mechanism of driver assembly 600 operates in the same manner as previously described in connection with driver assembly 100.

When a distally directed force is applied, via handle portion 108 of driver assembly 600, surgical fastener 602 may be inserted into a hole in the bone and flange portion 602c of surgical fastener 602 makes contact with an outer surface of the bone (or bone plate), thereby causing holding sleeve 622 pressing against flange portion 602c to be displaced in the proximal direction. Displacement of holding sleeve 622 in the proximal direction compresses front spring 620 communicatively coupled thereto. As front spring 620 is compressed, driver shaft 618 may emerge from a distal end of a cavity 622a provided in holding sleeve 622 to make contact with central pin member 602b. The impact force generated by the automatic trigger mechanism, as delivered through driver shaft 618, drives central pin member 602b in the distal direction, which in turn fully expands outer body 602a of surgical fastener 602 and secures it in the bone.

Whereas particular embodiments of the present invention are described in the foregoing description and illustrated in the accompanying drawings, it is to be understood that the present invention is not limited to the embodiments disclosed herein. It will be apparent to a person of ordinary skill in the art after having read the foregoing description that embodiments of the present invention are subject to alterations, modifications, rearrangements and substitutions without departing from the scope of the claims presented hereafter.

What is claimed is:

1. A driver assembly for affixing a surgical fastener, comprising:
a trigger mechanism body;
a receiver element communicatively coupled to a first spring, said receiver element and said first spring embodied within a first chamber portion of said trigger mechanism body;
a cam element communicatively coupled to a second spring, said cam element and said second spring embodied within a second chamber portion of said trigger mechanism body; and
a driver shaft embodied at least partially within said second chamber portion of said trigger mechanism body and communicatively coupled to a distal end of said cam element, said driver shaft extending externally from said trigger mechanism body in said distal direction.

2. The driver assembly of claim 1, further comprising a force adjustment mechanism permitting selection of a force setting to be associated with said drive spring.

3. The driver assembly of claim 1, wherein said receiver element comprises a bore portion shaped to receive a proximal end of said cam element.

4. The driver assembly of claim 3, wherein said second spring is adapted to keep said proximal end of said cam element out of alignment with said bore portion until said cam element is displaced to a position allowing it to be centered with said bore portion.

5. The driver assembly of claim 4, further comprising an internally tapered throat portion between said first chamber and said second chamber, said tapered throat portion aligning said cam element into said position allowing said cam element to be centered with said bore portion.

6. The driver assembly of claim 1, wherein said driver shaft is adapted for a linear motion along a longitudinal axis of said driver assembly.

7. The driver assembly of claim 1, wherein said driver shaft is adapted for a rotational motion about a longitudinal axis of said driver assembly.

8. The driver assembly of claim 7, wherein said driver shaft adapted for said rotational motion comprises at least one helical groove provided along an exterior surface of its body.

9. The driver assembly of claim 1, further comprising a driver tip coupled to a distal end of said driver shaft.

10. The driver assembly of claim 9, wherein said distal end of said driver shaft is adapted for temporarily securing a surgical fastener on said driver tip by means of a taper fit or an interference fit.

11. The driver assembly of claim 9, wherein said driver tip is adapted for use with a linearly insertable surgical fastener.

12. The driver assembly of claim 9, wherein said driver tip is adapted for use with a rotationally insertable surgical fastener.

13. A driver assembly for affixing a surgical fastener, comprising:
a handle portion;
an elongated neck portion embodied at least partially within said handle portion and extending externally from said handle portion in a distal direction, said distal end of said elongated neck portion coupled to a nose piece;
a receiver element embodied within said elongated neck portion and a drive spring embodied within said elongated neck portion, said receiver element communicatively coupled to said drive spring;
a cam element embodied at least partially within said nose piece and an alignment spring embodied within said nose piece, said cam element communicatively coupled to said alignment spring; and
a driver shaft embodied at least partially within said nose piece and extending externally from said nose piece in said distal direction.

14. The driver assembly of claim 13, further comprising a force adjustment mechanism permitting selection of a force setting to be associated with said drive spring.

15. The driver assembly of claim 13, wherein said receiver element comprises a bore portion shaped to receive a proximal end of said cam element.

16. The driver assembly of claim 15, wherein said alignment spring is adapted to keep said proximal end of said cam element out of alignment with said bore until said cam element is displaced to a position allowing it to be centered with said bore portion.

17. The driver assembly of claim 16, further comprising an internally tapered throat portion provided in said elongated neck portion, said tapered throat portion aligning said cam element into said position allowing said cam element to be centered with said bore portion.

18. The driver assembly of claim 13, wherein said driver shaft is adapted for a linear motion along a longitudinal axis of said driver assembly.

19. The driver assembly of claim 13, wherein said driver shaft is adapted for a rotational linear motion about a longitudinal axis of said driver assembly.

20. The driver assembly of claim 19, wherein said driver shaft cam element adapted for said linear rotational motion comprises at least one helical groove provided along an exterior surface of its body.

21. The driver assembly of claim 20, wherein said nose piece comprises at least one pin member perpendicular to said longitudinal axis of said driver assembly and positioned to protrude into said helical groove.

22. The driver assembly of claim 13, further comprising a driver tip coupled to a distal end of said driver shaft.

23. The driver assembly of claim 22, wherein said distal end of said driver shaft is adapted for temporarily securing a surgical fastener on said driver tip by means of a taper fit or an interference fit.

24. The driver assembly of claim 22, wherein said driver tip is adapted for a linearly insertable surgical fastener.

25. The driver assembly of claim 22, wherein said driver tip is adapted for a rotationally insertable surgical fastener.

26. A driver assembly for affixing a two-part surgical fastener, comprising:
a trigger mechanism body coupled to said handle portion;
a receiver element communicatively coupled to a first spring, said receiver element and said first spring embodied within a first chamber portion of said trigger mechanism body;
a cam element communicatively coupled to a second spring, said cam element and said second spring embodied within a second chamber portion of said trigger mechanism body;
a holding sleeve and a third spring slidably affixed to a distal end of said second chamber portion, said holding sleeve communicatively coupled to said third spring; and
a driver shaft embodied at least partially within said second chamber portion of said trigger mechanism body and communicatively coupled to a distal end of said cam element, said driver shaft extending externally from said trigger mechanism body in said distal direction and embodied at least partially within said holding sleeve.

27. An automatic trigger mechanism in a driver assembly for affixing a surgical fastener, comprising:
a receiver element coupled to a drive spring, said receiver element and said drive spring embodied within a first body space; and
a cam element coupled to an alignment spring, said cam element and said alignment spring embodied within a second body space, said second body space having an opening at a proximal end to allow a proximal end of said cam element to partially enter said first body space and communicate with said receiver element;
wherein said proximal end of said cam element displaces said receiver element, compressing said drive spring coupled thereto, upon application of a distally directed force; and
wherein an impact force is delivered by said drive spring upon said cam element being aligned to be received within a bore portion provided in said receiver element.

28. A method of affixing a surgical fastener to a target location, comprising:
positioning a surgical fastener provided on a tip of a driver assembly in said target location; and
applying a distally directed force to said driver assembly, wherein application of said distally directed force automatically triggers delivery of an impact force for seating said surgical fastener to said target location.
